(19) United States
(12) Patent Application Publication
(10) Pub. No.: US 2023/0241369 A1
(43) Pub. Date: Aug. 3, 2023

(54) Universal Caps for Medical Connectors

(71) Applicant: Becton, Dickinson and Company, Franklin Lakes, NJ (US)

(72) Inventors: Shishir Prasad, Ramsey, NJ (US); Prasad Govindaraj, Coimbatore (IN); Sridhaar Nandakumar, Chennai (IN); Balaji Kannan, Chennai (IN); Mohamed Shafiq, Bangalore (IN)

(21) Appl. No.: 18/103,034

(22) Filed: Jan. 30, 2023

(30) Foreign Application Priority Data
Jan. 31, 2022 (IN) 202211005267

(51) Int. Cl. A61M 39/20

(52) U.S. Cl. CPC A61M 39/20; A61M 2039/1083

(57) ABSTRACT

A cap configured to engage different types of connectors includes a housing, an absorbent support, and a seal. The housing includes a sidewall extending between a first end and a second end of the housing. The absorbent support is positioned in the housing. The seal is mounted to a portion of the absorbent support and is configured to cover an opening of a first connector or a second connector when one of the first connector or the second connector is inserted into the housing. The cap is configured to be secured to the first connector by an engagement between a portion of an inner surface of the sidewall of the housing and the first connector and to the second connector by an interference engagement between another portion of the inner surface of the sidewall of the housing and an outer surface of the second connector.

### CROSS-REFERENCE TO RELATED APPLICATION

The present application claims priority to Indian Patent Appl. No. 202211005267, entitled “Universal Caps for Medical Connectors” filed Jan. 31, 2022, the entire disclosure of which is hereby incorporated by reference in its entirety.

### BACKGROUND OF THE INVENTION

### Field of the Invention

The present disclosure relates generally to caps for medical connectors and, in particular, to a medical cap configured to be attached to at least two different types of connectors, such as to a male luer connector or a female luer connector.

### Description of Related Art

Vascular access devices (VADs) are commonly used medical devices, which can include intravenous (IV) catheters, such as peripheral catheters or central venous catheters. If not properly maintained or if exposed to a non-sterile environment, the VADs can become contaminated, sealed with blood clots, and/or can spread infection. Further, bacteria and other microorganisms may gain entry into a patient's vascular system from access hubs, ports, or valves upon connection to the VAD to deliver a fluid or pharmaceutical to a patient. Therefore, each access hub (or port/valve or connection) configured for attachment to a VAD is associated with some risk of transmitting a catheter related bloodstream infection (CRBSI) to a patient.

Many medical facilities implement sterile practices and protocols to ensure that VADs and access hubs or ports are used properly and do not become sealed or infected. These protocols often include sterilizing the access hubs, ports, and VADs, as well as flushing the catheter with a flush solution prior to use. Specifically, VAD standards of practice usually recommend flush procedures be performed after catheter placement, before fluid infusion, and before and after drug administration, blood sampling, transfusions, and/or administration of parenteral nutrition. Standards of practice can also require that access hubs, ports, and valves be capped with disinfection caps, when not in use, to prevent microbial ingress into the hub, port, or valve and to sterilize areas of the hub, port, or valve that contact the VAD. Disinfection caps are disposable cap devices that contain an amount of cleaning or disinfecting solution for sterilizing portions of the port, hub, and valve.

Access hubs and ports can have a variety of different types of male or female connectors for securing the hub or port to the VAD. Currently, practitioners often carry several types of caps with them so that they can cap the different types of hubs and ports that may be used for a particular patient. For example, caps for male needleless connectors and female needleless connectors, as well as intravenous (IV) and hemodialysis lines, use different connector designs and may require different caps. In particular, there can be “male disinfecting cap devices” for disinfecting ISO594-2 type of female threaded fluid luer connectors and “female disinfecting cap devices” for disinfecting ISO594-2 type of male threaded fluid luer connectors.

Some examples of universal caps that fit on both male and female connectors are known. For example, U.S. Pat. No. 10,871,246, entitled “Universal connector or cap for male and female threaded fittings,” which is incorporated herein by reference in its entirety, discloses a cap including a threaded protrusion that can engage both a male connector and a female connector. However, there is a need for simpler cap designs that can be manufactured inexpensively and efficiently. The universal caps of the present disclosure are configured to attach to both male and female medical connectors in a secure manner sufficient for preventing microbial ingress. Further, the universal caps of the present disclosure are configured to be easy to manufacture in a single-molding process.

### SUMMARY OF THE INVENTION

According to an aspect of the disclosure, a cap configured to engage at least a first connector and a second connector of different types includes a housing, an absorbent support, and a seal. The housing includes a first end, an open second end, and a sidewall extending between the first end and the second end. The absorbent support is positioned in the housing configured to contact portions of the first connector or the second connector inserted into the housing. The seal is mounted to a portion of the absorbent support and is configured to cover an opening of the first connector or the second connector when one of the first connector or the second connector is inserted into the housing. The cap is configured to be secured to the first connector by an engagement between a portion of an inner surface of the sidewall of the housing and the first connector and to the second connector by an interference engagement between another portion of the inner surface of the sidewall of the housing and an outer surface of the second connector.

According to another aspect of the disclosure, a cap configured to engage at least a first connector and a second connector of different types includes a housing, an absorbent support positioned in the housing, and a seal mounted to a portion of the absorbent support. The housing includes a first end, an open second end, a sidewall extending between the first end and the second end, at least one prong extending inwardly from an inner surface of the sidewall, and at least one ramp protrusion extending inwardly from the inner surface of the sidewall. The absorbent support positioned in the housing is configured to contact portions of the first connector or the second connector inserted into the housing. The seal mounted to a portion of the absorbent support is configured to cover an opening of the first connector or the second connector when one of the first connector or the second connector is inserted into the housing. The at least one prong is configured to engage threads of the first connector for securing the first connector to the cap. The at least one ramp protrusion is configured to form a friction engagement with the second connector for securing the second connector to the cap.

Non-limiting illustrative examples of embodiments of the present disclosure will now be described in the following numbered clauses.

Clause 1: A cap configured to engage at least a first connector and a second connector of different types, the cap comprising: a housing comprising a first end, an open second end, and a sidewall extending between the first end and the second end; an absorbent support positioned in the housing configured to contact portions of the first connector or the second connector inserted into the housing; and a seal mounted to a portion of the absorbent support configured to cover an opening of the first connector or the second connector when one of the first connector or the second connector is inserted into the housing, wherein the cap is configured to be secured to the first connector by an engagement between a portion of an inner surface of the sidewall of the housing and the first connector and to the second connector by an interference engagement between another portion of the inner surface of the sidewall of the housing and an outer surface of the second connector.

Clause 2: The cap of clause 1, wherein the first connector is a female luer connector comprising a threaded outer surface that engages the inner surface of the sidewall of the housing.

Clause 3: The cap of clause 2, wherein the inner surface of the sidewall comprises threads that engage the threaded outer surface of the female luer connector.

Clause 4: The cap of clause 2 or clause 3, wherein the housing is sized to receive the female luer connector having an outer diameter of at least 12.5 mm.

Clause 5: The cap of any of clauses 1-4, wherein the second connector comprises a male luer connector comprising a stem configured to be inserted into the absorbent support, such that the opening of the male luer connector is sealed by the seal mounted to the absorbent support.

Clause 6: The cap of clause 5, wherein the male luer connector further comprises an annular shield comprising a threaded inner surface extending about the stem.

Clause 7: The cap of clause 5 or clause 6, wherein the housing is sized to receive the male luer connector having an outer diameter of from 8.0 mm to 14.0 mm.

Clause 8: The cap of any of clauses 1-7, wherein the first end of the housing is closed comprising a wall extending over the sidewall of the housing.

Clause 9: The cap of any of clauses 1-8, wherein the sidewall of the housing comprises a flexible portion proximate to the second end of the housing configured to press against and deform to accommodate portions of the first connector or the second connector when the first connector or the second connector is inserted into the housing.

Clause 10: The cap of clause 9, wherein the flexible portion of the sidewall is configured to extend radially outwardly to accommodate portions of the first connector or the second connector having an outer diameter larger than an inner diameter of the sidewall in an unbiased state.

Clause 11: The cap of clause 9 or clause 10, wherein the flexible portion of the sidewall has a variable inner diameter that is widest at the second end of the housing and becomes narrower towards the first end of the housing.

Clause 12: The cap of any of clauses 9-11, wherein the flexible portion deforms to form an interference engagement with an outer surface of the second connector.

Clause 13: The cap of clause 11, wherein the flexible portion comprises a plurality of tiers having different inner diameters.

Clause 14: The cap of clause 13, wherein the sidewall for at least one of the tiers is tapered being wider near the open second end of the housing and narrower near the first end of the housing.

Clause 15: The cap of any of clauses 9-14, wherein the sidewall further comprises a rigid portion that does not deform when the first connector or the second connector is inserted into the housing.

Clause 16: The cap of clause 15, wherein the rigid portion of the sidewall is between the first end of the housing and the flexible portion of the sidewall.

Clause 17: The cap of clause 15 or clause 16, wherein the rigid portion comprises a threaded inner surface configured to engage a corresponding threaded outer surface of the first connector.

Clause 18: The cap of any of clauses 15-17, wherein the rigid portion comprises at least one axial ridge extending radially outwardly from an outer surface of the sidewall for providing rigidity for the rigid portion.

Clause 19: The cap of any of clauses 15-18, wherein the rigid portion comprises areas of increasing wall thickness and/or that are co-molded with a polymer that is more rigid than a material that forms other areas of the housing.

Clause 20: The cap of any of clauses 1-19, wherein the housing is a single-molded part.

Clause 21: The cap of any of clauses 1-20, wherein the housing comprises a rigid thermoplastic polymer, such as polyester, polycarbonate, polypropylene, polyethylene, polyethylene terephthalate, or acrylonitrile butadiene styrene.

Clause 22: The cap of any of clauses 1-21, wherein the absorbent support comprises a cylindrical member sized to be received within a substantially cylindrical cavity defined by a portion of the inner surface of the sidewall of the housing.

Clause 23: The cap of any of clauses 1-22, wherein the absorbent support comprises a porous foam comprising a thermoplastic elastomeric material.

Clause 24: The cap of any of clauses 1-23, wherein the absorbent support comprises a cavity extending axially inwardly from a second end surface of the absorbent support and wherein the seal is received within the cavity.

Clause 25: The cap of any of clauses 1-24, wherein insertion of the first or second connector into the cap causes the connector to contact the absorbent support, and wherein contact between the connector and the absorbent support mechanically removes objects from an outer surface of the connector.

Clause 26: The cap of any of clauses 1-25, further comprising a cleaning solution absorbed by the absorbent support.

Clause 27: The cap of clause 26, wherein the cleaning solution comprises an antimicrobial, anti-fungal, antibacterial, or antiviral composition.

Clause 28: The cap of clause 26 or clause 27, wherein the cleaning solution comprises isopropyl alcohol (IPA), such as about 70% IPA.

Clause 29: The cap of clause 28, wherein the cleaning solution further comprises from about 0.5% to about 3.5% chlorhexidine gluconate.

Clause 30: The cap of any of clauses 1-29, wherein the seal comprises a closed cell foam.

Clause 31: The cap of any of clauses 1-30, further comprising a removable protective cover attached to the open second end of the housing for protecting an interior of the housing and the absorbent support prior to use.

Clause 32: The cap of clause 31, wherein the protective cover comprises a film with adhesive on a first side of the film for removably mounting the first side of the film to the open second end of the housing.

Clause 33: The cap of clause 31 or clause 32, wherein the protective cover is substantially impervious to air to prevent the absorbent support from drying prior to use.

Clause 34: The cap of any of clauses 31-33, wherein the protective cover is connected to the open second end of the housing by heat sealing.

Clause 35: The cap of any of clauses 1-34, wherein the absorbent support is shaped to clean and/or disinfect a distal portion and outer surface of a female connector and a tip and outer surface of a stem and an inner surface of an annular shield of a male connector.

Clause 35: The cap of any of clauses 1-34, further comprising at least one ramp protrusion extending inwardly from the inner surface of the housing positioned to contribute to the engagement between the second connector and the cap.

Clause 36: The cap of clause 35, wherein the at least one ramp protrusion is configured to form a friction engagement with the second connector for securing the second connector to the cap.

Clause 37: The cap of clause 35 or clause 36, wherein the at least one ramp protrusion comprises a first ramp protrusion and a second ramp protrusion positioned about 180 degrees apart from the first ramp protrusion about an inner circumference of the sidewall of the housing.

Clause 38: The cap of any of clauses 35-37, wherein the at least one ramp protrusion is formed by an inwardly deformed portion of the sidewall, which provides an axially extending groove on an outer surface of the sidewall.

Clause 39: The cap of any of clauses 35-38, wherein the at least one ramp protrusion is deformable and configured to deform radially outward to accept second connectors having different outer diameters.

Clause 40: A cap configured to engage at least a first connector and a second connector of different types, the cap comprising: a housing comprising a first end, an open second end, a sidewall extending between the first end and the second end, at least one prong extending inwardly from an inner surface of the sidewall, and at least one ramp protrusion extending inwardly from the inner surface of the sidewall; an absorbent support positioned in the housing configured to contact portions of the first connector or the second connector inserted into the housing; and a seal mounted to a portion of the absorbent support configured to cover an opening of the first connector or the second connector when one of the first connector or the second connector is inserted into the housing, wherein the at least one prong is configured to engage threads of the first connector for securing the first connector to the cap, and the at least one ramp protrusion is configured to form a friction engagement with the second connector for securing the second connector to the cap.

Clause 41: The cap of clause 40, wherein the at least one ramp protrusion extends axially along the inner surface of the housing, the at least one ramp protrusion comprising a narrow side proximate to the second open end of the housing and a wide side opposite the narrow side.

Clause 42: The cap of clause 40 or clause 41, wherein the at least one ramp protrusion comprises a first ramp protrusion and a second ramp protrusion positioned about 180 degrees apart from the first ramp protrusion about an inner circumference of the sidewall of the housing.

Clause 43: The cap of any of clauses 40-42, wherein the at least one prong and the at least one ramp protrusion are axially aligned on the inner surface of the housing.

Clause 44: The cap of any of clauses 40-43, wherein the at least one ramp protrusion is formed by an inwardly deformed portion of the sidewall, which provides an axially extending groove on an outer surface of the sidewall.

Clause 45: The cap of any of clauses 40-44, wherein the at least one ramp protrusion is deformable and configured to deform radially outward to accept second connectors having different outer diameters.

Clause 46: The cap of any of clauses 40-45, wherein the first connector is a female luer connector comprising a threaded outer surface that engages the at least one prong of the housing.

Clause 47: The cap of any of clauses 40-47, wherein the second connector comprises a male luer connector comprising a stem configured to be inserted into the absorbent support, such that the opening of the male luer connector is sealed by the seal mounted to the absorbent support.

Clause 48: The cap of clause 47, wherein the male luer connector further comprises an annular shield comprising a threaded inner surface extending about the stem and an outer surface, and wherein the outer surface of the annular shield contacts the at least one ramp protrusion of the housing forming the friction engagement between the male luer connector and the cap.

### BRIEF DESCRIPTION OF THE DRAWINGS

FIG. 1A is a cross-sectional view of an exemplary male connector, as is known in the prior art.

FIG. 1B is an example of a female connector including a septum with a slit, as is known in the prior art.

FIG. 2 is an exploded perspective view of a universal cap for medical connectors, according to an aspect of the present disclosure.

FIG. 3A is a perspective view of a housing of the cap of FIG. 2, according to an aspect of the present disclosure.

FIG. 3B is a side view of the housing of FIG. 3A.

FIGS. 3C and 3D are cross-sectional views of the housing of FIG. 3A.

FIG. 4A is a cross-sectional view showing a universal cap attached to a female connector, according to an aspect of the present disclosure.

FIG. 4B is a cross-sectional view showing a universal cap attached to another example of a female connector, according to an aspect of the present disclosure.

FIG. 5A is a cross-sectional view showing a universal cap attached to a male connector, according to an aspect of the present disclosure.

FIG. 5B is a cross-sectional view showing a universal cap attached to another example of a male connector, according to an aspect of the present disclosure.

FIG. 6A is a perspective view of another example of a universal cap, according to an aspect of the present disclosure.

FIG. 6B is a cross-sectional view of the universal cap of FIG. 6A.

FIGS. 6C and 6D are cross-sectional views showing the universal cap of FIG. 6A connected to male connectors of different sizes.

FIG. 7A is a perspective view of another example of a universal cap, according to an aspect of the present disclosure.

FIG. 7B is a cross-sectional view of the universal cap of FIG. 7A connected to a female connector.

FIG. 8A is a perspective view of another example of a universal cap, according to an aspect of the present disclosure.

FIG. 8B is a cross-sectional view of the universal cap of FIG. 8A.

FIG. 9A is a perspective view of another example of a universal cap, according to an aspect of the present disclosure.

FIG. 9B is a perspective view of a bottom portion of the universal cap of FIG. 9A.

FIG. 9C is a perspective view of a cross-section of the universal cap of FIG. 9A.

FIG. 10A is a perspective view of a cross-section showing the universal cap of FIG. 9A connected to a female connector.

FIG. 10B is a perspective view of a cross section showing the universal cap of FIG. 9A connector to a male connector.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

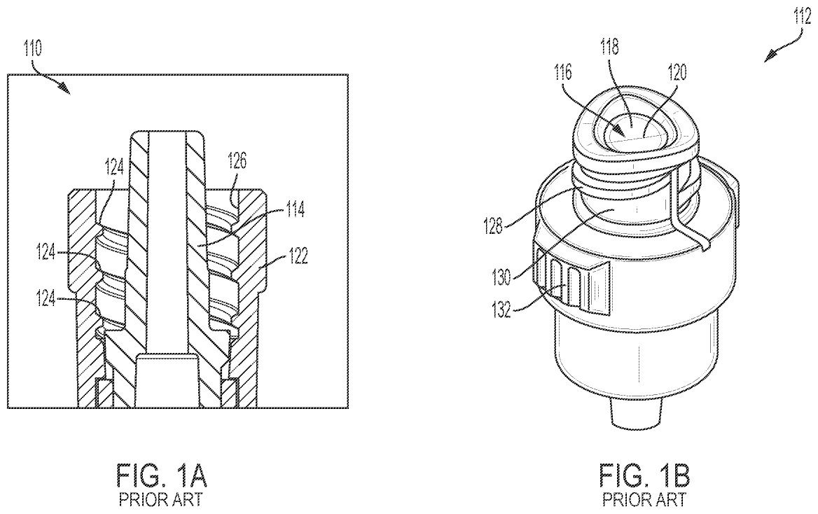

With reference to the figures, the present disclosure is directed to caps 10, 10b, 10c, 10d, 210 configured to be connected to a medical connector 110, 112, such as an access hub, port, or valve for a VAD, to prevent the connector, port, or VAD from being contaminated by, for example, microbes, debris, or other contaminants. In some cases, the caps 10, 10b, 10c, 10d, 210 can be configured to clean or disinfect the connector 110, 112 or port, ensuring that the connector 110, 112 or port remains sterile prior to use. The caps 10, 10b, 10c, 10d, 210 can be configured to remain in place on a connector 110, 112 or port for at least seven days, which is a maximum time of recommended use permitted by many medical facility sterile practice guidelines. The caps 10, 10b, 10c, 10d, 210 can be configured to engage or be connected to different sizes, configurations, or types of medical connectors 110, 112. For example, the caps 10, 10b, 10c, 10d, 210 can be configured to engage or be connected to both a male connector 110 and a female connector 112. As used herein, a “male connector” refers to a connector 110 comprising an elongated member, such as a tubular member or stem 114, configured to be inserted into a tube or opening having an inner diameter that is larger than an outermost diameter of the male connector 110. An exemplary male connector 110 is shown in FIG. 1A. A “female connector” refers to a connector 112 comprising an opening or port 116 that is configured to receive an elongated member or tubular member of another object or device in order to connect the object or device to the female connector 112. The female connector 112 can have a cover or septum 118 over the opening 116. An exemplary female connector 112 including a septum 118 with a slit 120 is shown in FIG. 1B.

In some examples, the caps 10, 10b, 10c, 10d, 210 of the present disclosure are configured to engage both a male luer connector 110 and a female luer connector 112. For example, the caps 10, 10b, 10c, 10d, 210 can be configured to receive a female luer connector 112 having an outer diameter of at least 12.5 mm. The caps 10, 10b, 10c, 10d, 210 can also be sized to receive a male luer connector 110 having an outer diameter of from 8.0 mm to 14.0 mm. As used herein, a “luer connector” refers to a connector that includes a tapered portion (i.e., a luer taper) for creating a friction engagement between a tapered stem 114 or elongated member of a male luer connector 110 and a tapered cavity. Specifically, the male luer connector 110 includes a tapered stem 114 or elongated member having a tapered outer surface. The female luer connector 112 can include a tapered cavity configured to receive and engage the tapered stem 114 or elongated member to connect the male luer connector 110 to the female luer connector 112.

In order to secure the male and female connectors 110, 112 together, in some examples, the connectors 110, 112 can include engaging structures, such as threads, for drawing the connectors 110, 112 together. For example, as shown in FIG. 1A, the male luer connector 110 can include an annular shield 122 extending about the tapered stem 114 or elongated member. The annular shield 122 can include threads 124 on an inner surface 126 of the shield 122 configured to engage corresponding threads 128 on an outer surface 130 of the female luer connector 112. For example, as shown in FIG. 1B, the female luer connector 112 includes the threads 128 extending from the outer surface 130 positioned to engage the threads 124 on the inner surface 126 of the annular shield 122 of the male luer connector 110. Twisting the female connector 112 relative to the male connector 110 causes the corresponding threads 124, 128 to engage, which draws the connectors 110, 112 together, such that the tapered stem 114 or elongated member of the male luer connector 110 moves through the opening 116 of the female connector 112. In some examples, the female connector 112 can also include vertical ribs 132 near a proximal end of the female connector 112, which can be used to manipulate the female connector 112 making it easier to twist the female connector 112 relative to another connector or device. As previously described, the caps 10, 10b, 10c, 10d, 210 of the present disclosure are configured to cover both the male luer connector 110 (shown in FIG. 1A) and the female luer connector 112 (shown in FIG. 1B).

Universal Cap with Internal Threads and Friction Engagement Structure

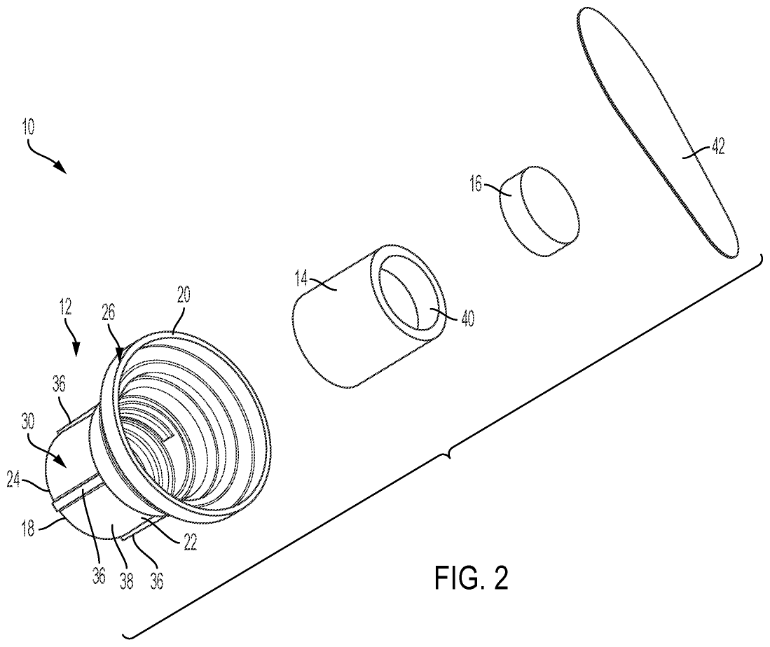

With reference to FIG. 2, in some examples, the cap 10 comprises a housing 12. The cap 10 further comprises an absorbent support 14 positioned in the housing 12 configured to contact portions of the male connector 110 or the female connector 112 inserted into the housing 12. For example, the cap 10 can be configured such that when the cap 10 is attached to the male luer connector 110, the stem 114 or elongated member of a male luer connector 110 inserts into the absorbent support 14 to form an interference engagement with the absorbent support 14. As described in further detail herein, a bottom portion of the housing 12 can also engage a corresponding portion of the male connector 110 to form a friction engagement between the housing 12 and the connector 110. The interference and/or friction engagement can secure the cap 10 to the male luer connector 110. The cap 10 can further comprise a seal 16 mounted to a portion of the absorbent support 14 configured to cover an opening or lumen of the male connector 110 or the female connector 112 when the male connector 110 or the female connector 112 is inserted into the housing 12. For example, when the male luer connector 110 is inserted in the housing 12, the seal 16 can contact and cover an opening at a distal end of the stem 114 or elongated member of the male luer connector 110, which seals the male luer connector 110. As described in further detail herein, the seal 16 can prevent fluids, such as cleaning or disinfecting solution in the cap 10, from flowing into the lumen of the male luer connector 110.

The cap 10 is configured to engage a variety of different configurations and orientations of medical connectors 110, 112. For example, the cap 10 can be configured to be secured to the female connector 112 by either an interference/friction engage or a threaded engagement between an inner surface of the housing 12 and a portion of the female connector 112. The cap 10 can also be configured to be secured to the male connector 110 by the interference or friction engagement between the outer surface 130 of the male connector 110 and the bottom portion of the housing 12 and/or the absorbent support 14. Thus, the cap 10 of the present disclosure is configured to be connected or engaged to both a female connector 112, such as a female luer connector, and to a male connector 110, such as a male luer connector, with different portions or components of the cap 10 engaging the different connector types.

There are numerous different commercially available medical devices, such as hubs, ports, and valves, which include different variations of male or female connectors 110, 112. The cap 10 of the present disclosure is configured to adapt or deform so that it can be secured to many or most of the numerous different types and sizes of connectors. For example, the cap 10 can be configured to attach to both male and female luer connectors, such as male or female Luer-Lok™ connectors by Becton Dickinson and Company. The cap 10 can also be configured to cover different connector designs including, without limitation, the BD Q-Syte™, BD MaxZero™, BD MaxPlus™, and SmartSite™ needle free connectors by Becton Dickinson and Company. The caps 10 can also be configured to be connected to male and/or female connectors by other manufactures including, without limitation, MicroClave® connectors (ICU Medical Inc.) and Ultrasite® connectors (B. Braun Medical Inc.). In other examples, the cap 10 can be configured to be connect to one or more of the following commercially available male connectors: Kendall 2001NP; BD MP5303-C; ICU Med 12664-28; RyMed RYM-5307HPU; B. Braun 470108; Baxter 2C8537; Kawasumi IV-0094; Zyno B2-70071-D; B. Braun 470124; Baxter 2C7462; and Smith's Medical 536035.

In some examples, the housing 12 of the cap 10 can be a molded part formed from a thermoplastic material, such as polyester, polycarbonate, polypropylene, polyethylene, polyethylene terephthalate, or acrylonitrile butadiene styrene. In some examples, the cap 10 is formed from a durable material, such as a material having a shore hardness D value of less than or equal to 60 shore D. Alternatively, the cap 10 can be formed from a more flexible material, such as a material having a shore hardness A value less than or equal to 80 shore A.

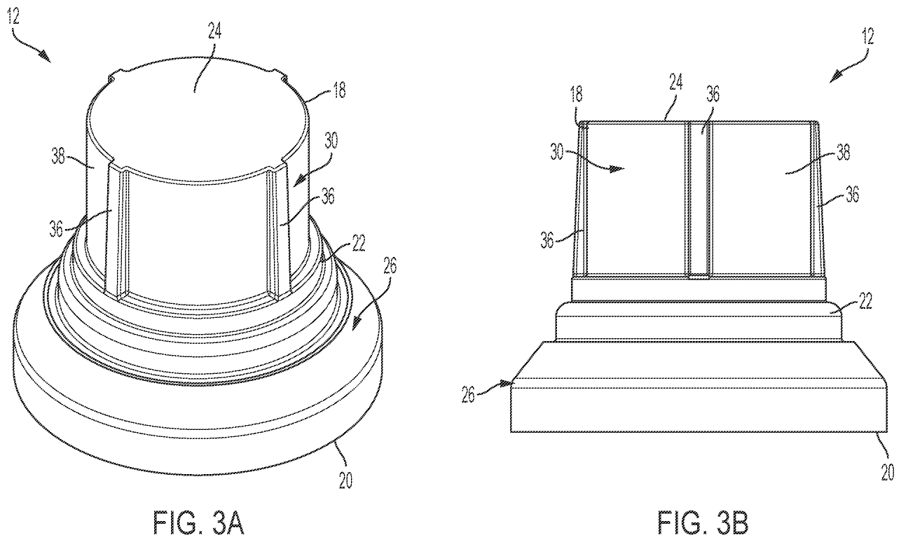

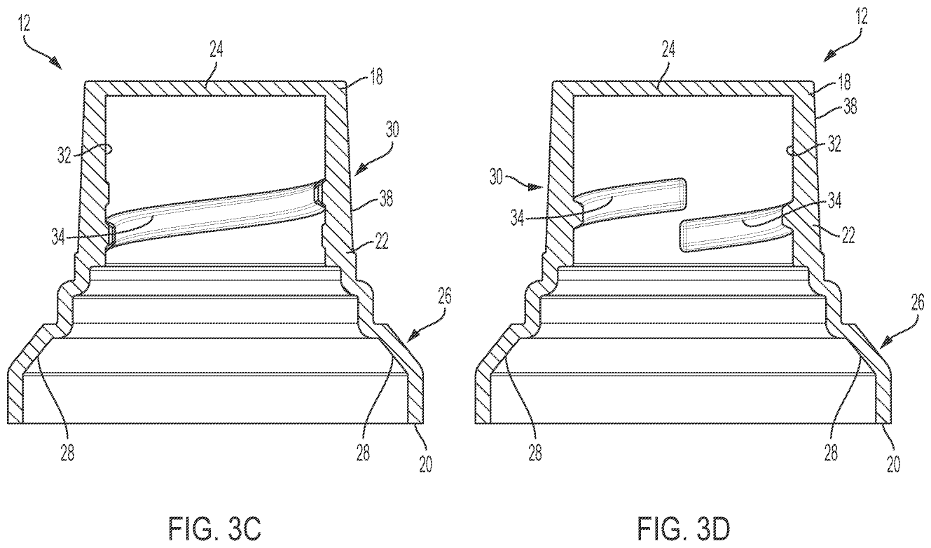

In some examples, the housing 12 is formed as a single integral and/or molded part. For example, the housing 12 can be formed by an injection molding process, where the entire housing 12 is formed in a single mold. The housing 12 can comprise a first or top end 18, a second or bottom end 20, and a sidewall 22 extending between the top end 18 and the bottom end 20 of the housing 12. In order to cover or enclose the connector 110, 112, port, or hub, the housing 12 can also include a top wall 24 extending over the top end 18 of the housing 12, which forms an elongated structure comprising a closed top end 18 and an open bottom end 20, as shown in FIGS. 3A-3D.

In some examples, the sidewall 22 of the housing 12 includes a flexible portion 26 proximate to the bottom end 20 of the housing 12 configured to press against and deform to accommodate portions of the female connector 112 or the male connector 110 when the connector 110, 112 is inserted into the housing 12. For example, the flexible portion 26 of the sidewall 22 can be configured to extend radially outwardly to accommodate portions of the connectors 110, 112 having an outer diameter larger than an inner diameter of the sidewall 22 of the housing 12 in an unbiased state. As used herein, an “unbiased state” refers to an orientation of the cap 10 when no external forces are applied to the cap 10 and when the cap 10 is not connected to a connector device or port. The flexible portion 26 can also be configured to bend, compress, or deform to adopt a curvature of a distal end portion of the connector 110, 112, hub, or port, such that the bottom end 20 of the cap 10 becomes curved when inserted onto the connector 110, 112, hub, or port. Further, the flexible portion 26 of the sidewall 22 can be configured to deform to form the friction engagement with the male connector 110 to secure the cap 10 to the male connector 110.

As previously described, the housing 12 can be made in a single molding process meaning that the flexible portion 26 of the housing 12 can be formed from the same material as other more rigid areas of the housing 12. In that case, the flexible portion 26 of the housing 12 can be molded to be thinner than other areas of the housing 12, thereby providing the flexible portion 26 with sufficient flexibility to deform to conform to a shape of and/or to engage the connector 110, 112, hub, or port.

In some examples, the flexible portion 26 of the sidewall 22 has a variable inner diameter to accommodate connectors 110, 112 of different sizes. For example, the flexible portion 26 can be widest at the bottom end 20 of the housing 12 and can become narrower moving axially through the housing 12. In some examples, some portion or the entire flexible portion 26 can be tapered, having an inner cavity with an angled or frusto-conical surface. In other examples, the flexible portion 26 can be tiered, such that an inner diameter of the flexible portion 26 decreases moving axially through the housing 12, but in a non-continuous manner. For example, the flexible portion 26 of the sidewall 22 can include three or more tiers, each having different inner diameters. Further, in some examples, one or more of the tiers includes an annular tapered surface 28, as shown in FIGS. 3C and 3D.

In some examples, the flexible portion 26 of the cap 10 can include tiers configured to engage an annular shield 122 (shown in FIG. 1A) of different commercially available male connectors. For example, the different tiers of the flexible portion 26 can have an inner diameter that matches an outer diameter of the annular shield of the commercially available male connectors. Many commercially available male connectors have an annular shield with an outer diameter of either 9.4 mm or 10.4 mm. For example, male connectors by RyMed, B. Braun, Zyno, and Smith's Medical often have an outer diameter of about 9.4 mm. By contrast, male connectors by Kendall, Becton Dickinson, ICU Medicals, Baxter, and Kawasumi often have an outer diameter of about 10.4 mm. Accordingly, the cap 10 have the present disclosure can have a first tier adapted to form an interference engagement with an annular shield having an outer diameter of 10.4 mm and a second tier adapted to form an interference engagement with an annular shield having an outer diameter of 9.4 mm. In some examples, the cap 10 can also include a third tier adapted, for example, to form an interference engagement with a portion of a female connector, such as with portions of the BD Q-Syte™ connector by Becton Dickinson. In particular, the third tier can have an inner diameter of about 13.4 mm or about 13.5 mm to form an interference engagement with the vertical ribs 132 of the female connector 112 shown in FIG. 1B.

In some examples, the sidewall 22 of the cap 10 also includes a rigid portion 30 that does not deform when the male connector 110 or the female connector 112 is inserted onto the housing 12. As shown in FIGS. 3A-3D, the rigid portion 30 is a substantially cylindrical structure extending between the closed top end 18 of the housing 12 and the flexible portion 26 of the sidewall 22. The rigid portion 30 can be formed from the same material as the flexible portion 26, but can be thicker to resist deformation. In some examples, the rigid portion 30 includes an inner surface 32 with threads 34 configured to engage threads 128 on the outer surface 130 of the female connector 112. For example, the threads 34 can make one or more complete turns about the inner surface 32 of the rigid portion 30 depending about a height of the rigid portion 30. In some examples, the threads 34 may make at least two complete turns about the inner surface 32 of the rigid portion 30 in order to ensure that the cap 10 can be used with female connectors 112 of various heights. As previously described, an exemplary female connector 112 including threads 128 protruding from the outer surface 130 of the connector 112, which can engage the threads 34 on the inner surface 32 of the housing 12, as shown in FIG. 1B. In other examples, the inner surface 32 of the rigid portion 30 can be smooth, without threads. In that case, the cap 10 can be configured such that an interference engagement is formed between the inner surface 32 of the rigid portion 30 and the female connector 112,

In some examples, the rigid portion 30 further includes axial ridges 36 extending outwardly from an outer surface 38 of the sidewall 22 for providing rigidity for the rigid portion 30. The axial ridges 36 may also make the cap 10 easier to grip and manipulate for users. For example, as shown in FIG. 2A, the rigid portion 30 includes four axial ridges 36 positioned at the 0 degree, 90 degree, 180 degree, and 270 degree positions about a periphery of the rigid portion 30 of the cap 10. In other examples, the rigid portion 30 can include fewer than four axial ridges 36 or more than four axial ridges 36. In some examples, the rigid portion 30 includes six axial ridges 36. Alternatively or in addition, the rigid portion 30 can include areas that are co-molded with a more rigid polymer to increase rigidity of the rigid portion 30.

With reference again to FIG. 2, the absorbent support 14 of the cap 10 can be a cylindrical structure or member sized to be received within a substantially cylindrical cavity defined by the inner surface 32 of the rigid portion 30. The absorbent support 14 can be held within the cylindrical cavity by conventional adhesives or fasteners, as are known in the art. In other examples, the absorbent support 14 can be held in place by friction between the inner surface 32 of the rigid portion 30 and the absorbent support 14.

The absorbent support 14 is formed from an absorbent material capable of absorbing a cleaning or disinfecting solution for cleaning and/or disinfecting portions of the male connector 110 and the female connector 112. In particular, the absorbent support 14 can be shaped and configured to clean and/or disinfect a distal or top portion of a female connector, including the septum 118 and slit 120 shown in FIG. 1B, as well as an outer surface of the tubular portion of the female connector 112. The absorbent support 14 can also be shaped and configured to clean and/or disinfect the tip and sides of the stem 114, as well as the inner surface 126 and threads 124 of the annular shield 122 of the male connector 110 (shown in FIG. 1A).

In some examples, the material of the absorbent support 114 can be abrasive with sufficient texture, friction, and/or anti-slip properties to scrub surfaces of the connectors 110, 112 to mechanically remove microbes, debris, and other contaminants from surfaces of the connectors 110, 112. The absorbent support 14 may also have sufficient texture to create an interference engagement with the stem 114 or elongated member of the male connector 110 when the male connector 110 is inserted into the housing 12. For example, the absorbent support 14 can comprise a thermoplastic elastomer, such as polypropylene, polyethylene, or synthetic or natural rubber (e.g., isoprene).

In some examples, the absorbent support 14 can be a porous foam or sponge capable of absorbing the cleaning or disinfecting solution. For example, the foam material can be a Plastazote® foam, which is an engineered polymer foam by Zotefoams PCL. Desirably, a porosity of the absorbent support 14 should be optimized so that the material is abrasive enough to scrub or mechanically remove objects from surfaces of the connectors 110, 112, while, at the same time, limiting ingress of cleaning or disinfecting solution into portions of the connectors 110, 112. Further, a height of the absorbent support 14 and/or amount of cleaning or disinfecting solution contained therein can be optimized for use with both short and tall connectors 110, 112. As used herein, a “short connector” refers to a connector that does not insert very far into the cap 10. A “tall connector” refers to a connector that inserts into the cap 10 by a substantial distance, such that a distal end of the connector is proximate to the top wall 24 of the housing 12. In particular, the height of the absorbent support 14 and amount of cleaning solution contained therein should be large enough so that sufficient cleaning solution is released from the absorbent support 14 when the cap 10 is attached to a short connector to disinfect surfaces of the short connector. However, the height of the absorbent support 14 and amount of cleaning solution may be somewhat limited so that liquid ingress into a lumen of the connector 110, 112 does not occur when the cap 10 is attached to a taller connector.

As previously described, the absorbent support 14 is provided (e.g., presoaked) with the cleaning or disinfecting solution. For example, the cleaning or disinfecting solution can be an antimicrobial, anti-fungal, antibacterial, or antiviral solution that cleans and sterilizes surfaces of the connectors 110, 112. In some examples, the cleaning solution can be isopropyl alcohol (IPA), such as about 70% IPA. In other examples, the cleaning solution can be about 0.5% to about 3.5% chlorhexidine gluconate in combination with about 70% IPA. A chlorohexidine composition may be beneficial because it has a slower evaporation rate than IPA and, therefore, provides a more persistent disinfectant activity alter the cap 10 is removed from the connector 110, 112 and before the VAD is connected to the hub, port, or valve.

In some examples, the absorbent support 14 includes a cavity 40 extending axially inwardly from an end surface of the absorbent support 14. For example, the cavity 40 can be a cylindrical cavity. The cavity 40 can be sized to receive the seal 16 so that the seal 16 is correctly positioned to contact and seal an end of the connector 110, 112, when the connector 110, 112 is inserted into the housing 12. The seal 16 can be formed from a material that prevents fluid, such as the cleaning solution, from entering the connector 110, 112. For example, the seal 16 can comprise a non-porous closed cell foam that is denser and/or more rigid than the foam used for the absorbent support 14.

As shown in FIG. 2, the cap 10 can also include a removable protective cover 42 attached to the open bottom end 20 of the housing 12 for protecting an interior of the housing 12 and the absorbent support 14 contained therein prior to use. The protective cover 42 can comprise a sheet, such as a polymer film, with adhesive on a first side of the sheet for removably mounting the protective cover 42 to the open bottom end 20 of the housing 12. Alternatively, the protective cover 42 can be removably mounted to the bottom end 20 of the cap 10 by heat sealing. The protective cover 42 can be slightly larger than the bottom end 20 of the housing 12, so that the cover 42 can be easily grasped by the practitioner to remove the protective cover 42 from the cap 10 prior to use. The protective cover 42 can be formed from a material that is impervious or substantially impervious to air, so that the cleaning solution on the absorbent support 14 does not evaporate or dry-out prior to use of the cap 10. Accordingly, the protective cover 42 increases a shelf life of the cap 10, as well as prevents microbes and other debris from collecting in the cap 10 prior to use.

The cap 10 can be provided in a number of different packages or containers, as are known in the art. For example, caps 10 can be packaged individually in paper or plastic packages. In other examples, multiple caps 10 can be provided together in a single packaging. For example, multiple caps 10 can be provided on strips of paper or plastic film configured to be hung from, for example, an IV pole. Multiple caps 10 can also be packaged in blisters or flow wrap on a strip or card to protect the caps if dropped prior to use.

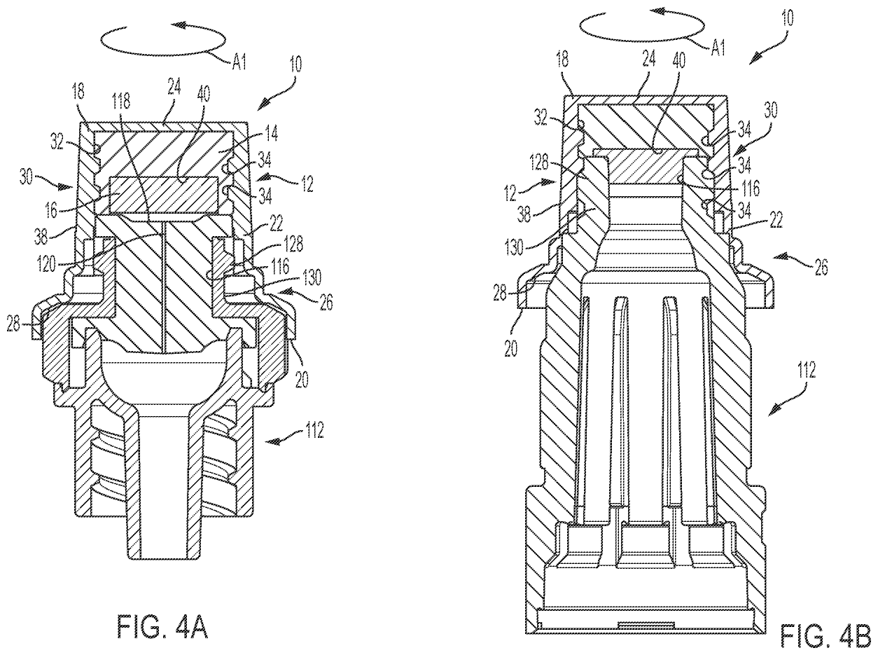

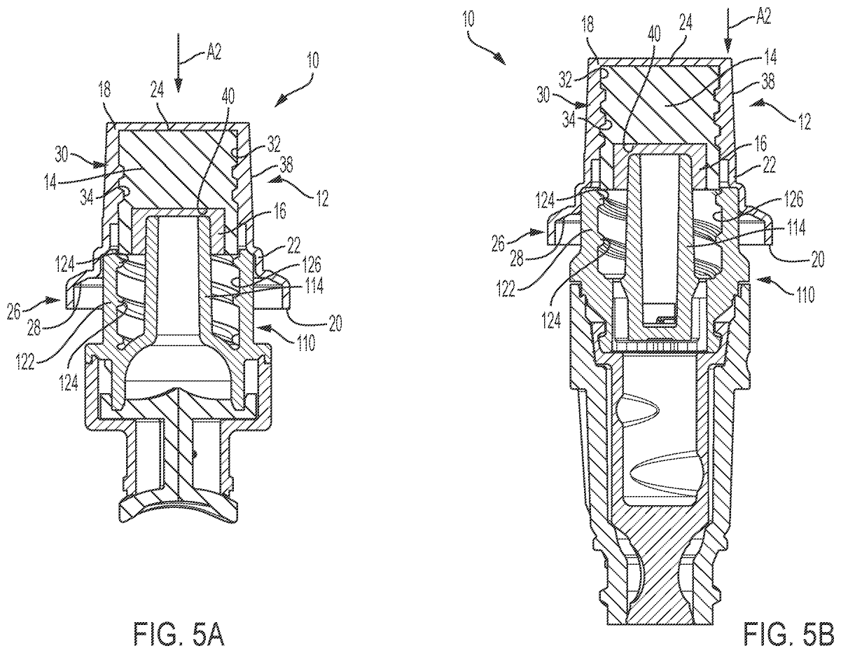

As previously described, the cap 10 of the present disclosure is configured to be connected to a female connector 112 or a male connector 110. FIGS. 4A and 4B show the cap 10 attached to different examples of female connectors 112. FIGS. 5A and 5B show the cap 10 connected to different examples of male connectors 110. Specifically, in FIG. 4A, the cap 10 is connected to a female connector 112 including a septum 118 with a slit 120, such as the BD Q-Syte™ connector, by Becton Dickinson and Company. An exemplary female connector 112 including the septum 118 and slit 120 is described in U.S. Pat. No. 7,806,890, entitled “Vascular access device volume displacement”, which is incorporated by reference herein in its entirety. FIG. 4B shows the cap 10 attached to a BD MaxPlus™ connector by Becton Dickinson and Company.

In order to connect the cap 10 to a female connector 112, the practitioner first removes any packaging from the cap 10 and removes the protective cover 42 from the bottom end 20 of the housing 12. The cap 10 is then inserted over the female connector 112 to bring the threads 128 of the female connector 112 into contact with the threads 34 on the inner surface 32 of the sidewall 22 of the housing 12. As previously described, the flexible portion 26 of the sidewall 22 may deform to accommodate the size and shape of the female connector 112. Once the threads 128 of the female connector 112 are in contact with the threads 34 of the housing 12, the practitioner rotates the cap 10 about the female connector 112, as shown by arrow A1 in FIGS. 4A and 4B, in order to secure the cap 10 to the connector 112. Rotation of the cap 10 can cause the absorbent support 14 to compress and/or to release the cleaning solution, which contacts surfaces of the female connector 112 to sterilize the surfaces of the connector 112. In particular, the cleaning solution may contact and sterilize portions of the septum 118 and outer surface 130 of the female connector 112. In order to remove the cap 10, the practitioner rotates the cap 10 in an opposite direction, causing threads 34 of the housing sidewall 22 to release from the threads 128 of the female connector 112. Once the threads 34, 128 are released, the practitioner can pull the cap 10 away from the female connector 112.

FIG. 5A shows a male BD Q-Syte™ connector 110 inserted over or attached to the cap 10. FIG. 5B shows a male BD MaxZero™ connector 110 inserted over or attached to the cap 10. In order to connect the cap 10 to a male connector 110, the practitioner first removes any packaging and the protective cover 42 from the cap 10. The practitioner then pushes the cap 10 onto the male connector 110 in a direction of arrow A2 (shown in FIGS. 5A and 5B). The stem 114 of the male connector 110 contacts and is sealed by the seal 16 enclosed within the housing 12 of the cap 10. Further, contact between the male connector 110 and the seal 16 or absorbent support 14 creates an interference engagement that secures the cap 10 to the male connector 110. Also, contact between an outer surface of the male connector 110 and the flexible portion 26 of the sidewall 22 of the housing 12 can form an interference or friction engagement between the male connector 110 and the cap 10 for securing the cap 10 to the male connector 110. As previously described, the contact between the male connector 110 and the absorbent support 14 can also release cleaning solution, causing the cleaning solution to contact and sterilize surfaces of the male connector 110. In particular, the cleaning solution can contact both an outer surface of the stem 114 of the male connector 110 and the inner surface 126 of the annular shield 122 of the male connector 110, thereby disinfecting and sterilizing both surfaces 114, 126. Finally, direct contact between surfaces of the male connector 110 and abrasive portions of the absorbent support 14 or seal 16 can mechanically remove microbes, debris, and other contaminants from surfaces of the male connector 110, contributing to the disinfecting effects provided by the cap 10. In order to remove the cap 10 from the male connector 110, the practitioner grasps the cap 10 and pulls it away from the male connector 110 with sufficient force to overcome the interference and/or friction engagement between the male connector 110 and portions of the cap 10. Once the cap 10 is removed, a VAD can be connected to the hub, port, or valve through the male connector 110, as previously described.

### Universal Cap for Connection to Connectors of Different Sizes

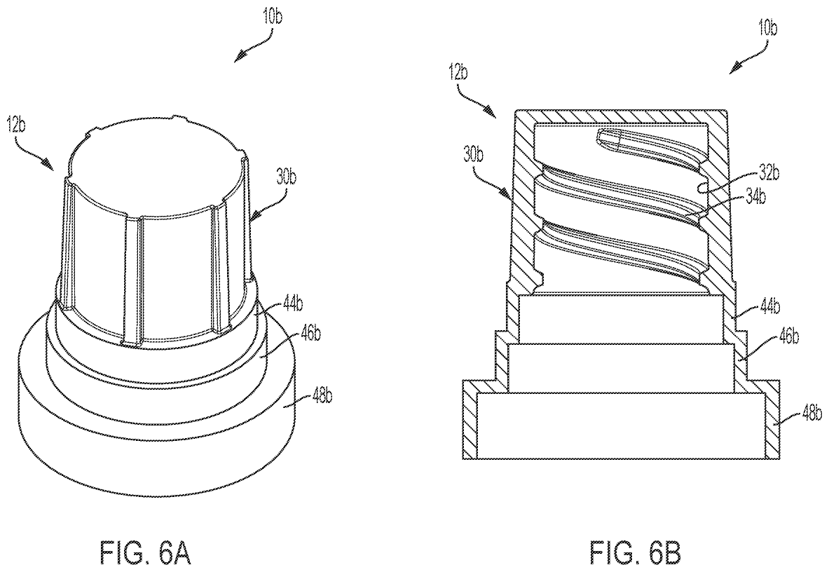

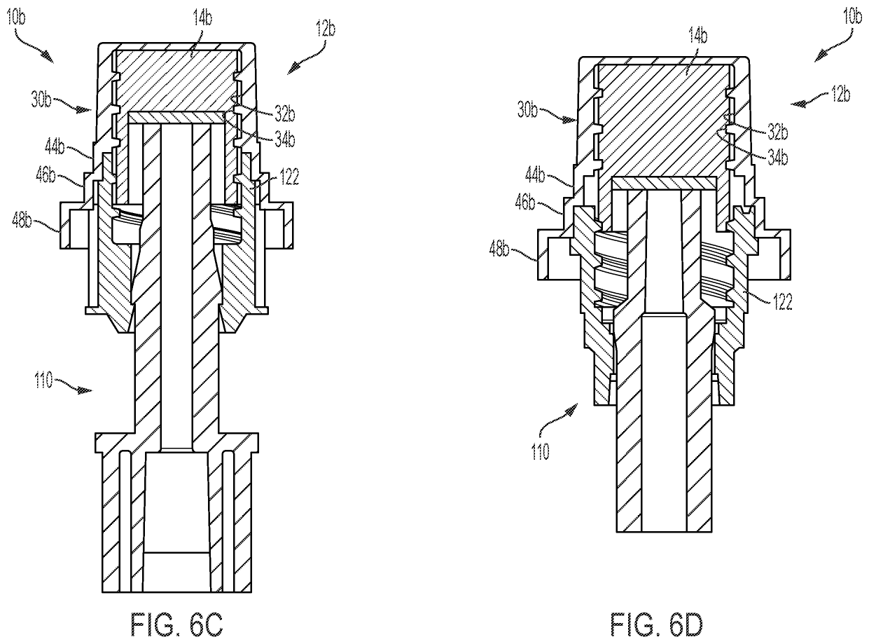

FIGS. 6A-8B show additional examples of caps 10b, 10c, 10d configured to be connected to different types or sizes of connectors, such as to a male connector 110 and/or a female connector 112. For example, FIGS. 6A-6D show a cap 10b including a housing 12b and absorbent support 14b. The housing 12b comprises an elongated rigid portion 30b compared to the rigid portion 30 of the previously described cap 10 (shown in FIGS. 2A-5B). The elongated rigid portion 30b of the cap 10b includes threads 34b that make two full turns about the inner surface 32b of the housing 12b. Further, the cap 10b includes three tiers 44b, 46b, 48b sized to receive different sizes of connectors. Specifically, as shown in FIG. 6C, a narrowest tier 44b can receive a male connector 110 having a narrow annular shield 122. As shown in FIG. 6D, a middle tier 46b can receive a male connector 110 with a wider annular shield 122. The widest or bottom tier 48b can be sized to receive a female connector 112, such as a female connector including vertical ridges 132 (shown in FIG. 1B).

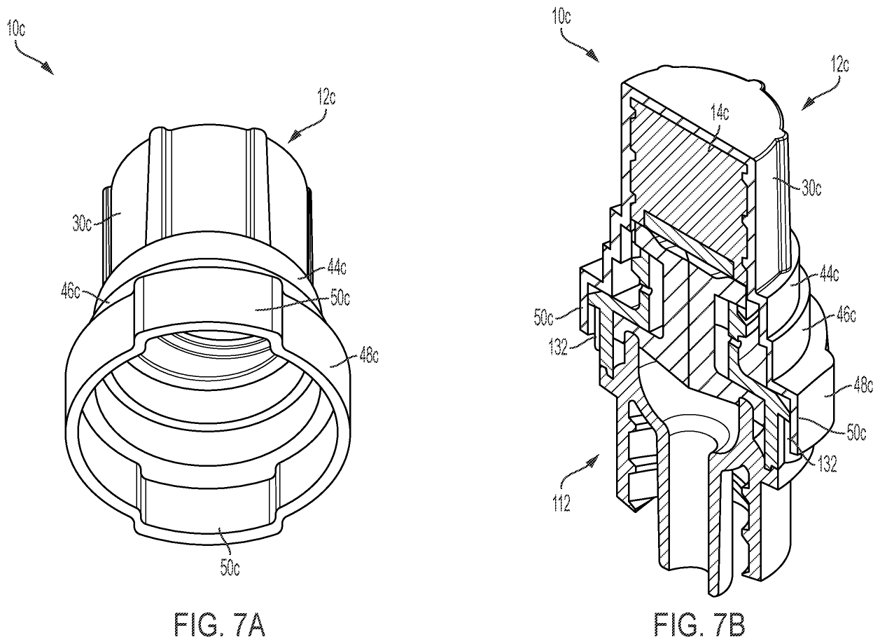

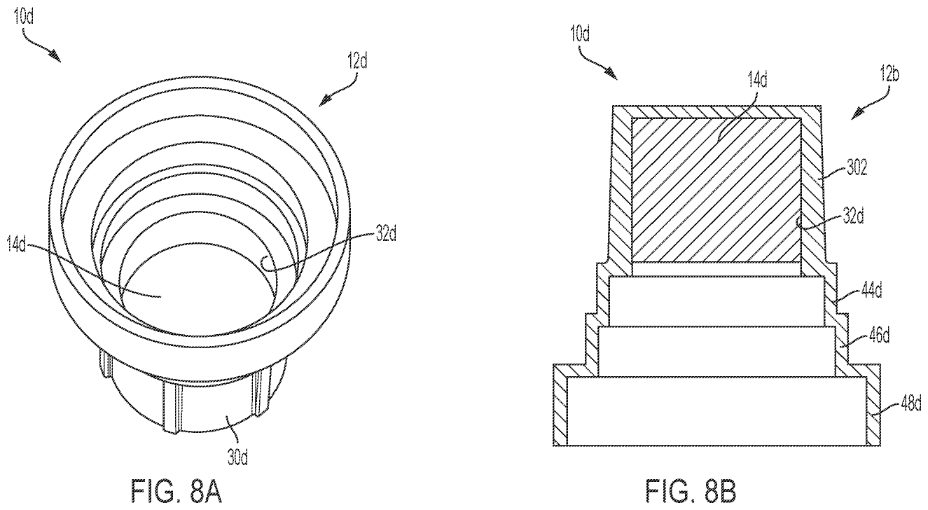

FIGS. 7A and 7B show another example of a universal cap 10c including a housing 12c comprising a rigid portion 50c and multiple tiers (e.g., a narrowest tier 44c, a middle tier 46c, and a widest tier 48c) and an absorbent support 14c. The cap 10c is configured to be connected to both male connectors 110 and female connectors 112 and, in particular, to a female connector 112 including vertical ribs 132 (shown in FIG. 1B). In order to accommodate female connectors 112 with the vertical ribs 132, the cap 10c includes bump outs, which can also be referred to as radially extending guides 50c. As shown in FIGS. 7A and 8B, the radially extending guides 50c are positioned in the widest tier 48c of the housing 12c. For example, the widest tier 48c can include guides 50c spaced about 180 degrees apart about the circumference of the housing 12c. As shown in FIG. 7B, the guides 50c are sized to receive vertical ribs 132 of a female connector 112 (shown in FIG. 1B).

FIGS. 8A and 8B show another example of a cap 10d including a housing 12d and absorbent support 14d (shown in FIG. 8B). The housing 12d comprises a rigid portion 30d with a smooth inner surface 32d. By contrast, in previous examples, the inner surface 32 of the rigid portion 30 included the threads 34 for engaging the female connector 112. The housing 12d also includes the multiple tiers 44d, 46d, 48d, as in previous examples. The cap 10d connects to a male connector 110 in a similar manner as in previous examples. Specifically, the cap 10d forms a friction engagement between the annular shield 122 of the cap 10d and inner surfaces of one of the tiers 44d, 46d, 48d of the housing 12d. Unlike in previous examples, the cap 10d of FIGS. 8A and 8B is also configured to form an interference or friction engagement with female connectors 112 of various shapes and sizes to secure the cap 10d to the female connector. For example, the cap 10d can form an interference engagement with portions of the female connector 112 that insert into the rigid portion 30d of the housing 12d. A friction engagement can also be formed between outer surfaces of the female connector 112 and inner surfaces of one of the tiers 44d, 46d, 48d of the housing 12d. Because the cap 10d of FIGS. 8A and 8B does not attach to the female connector 112 with threads, the cap 10d can be used with various types of female connectors 112 that do not include threads.

Universal Cap with Ramp Protrusion(s) for Friction Engagement with Male Connectors

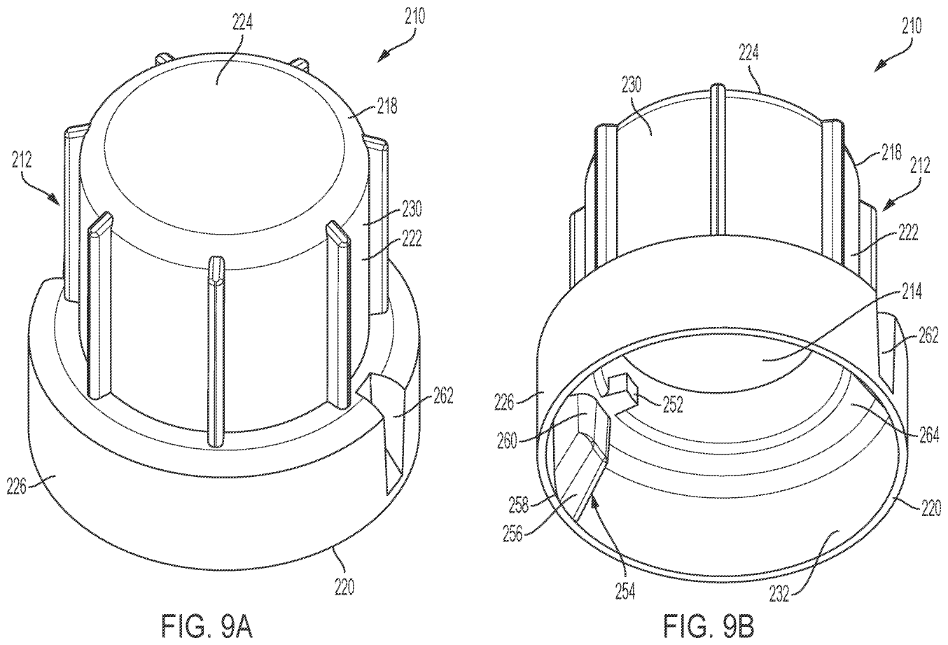

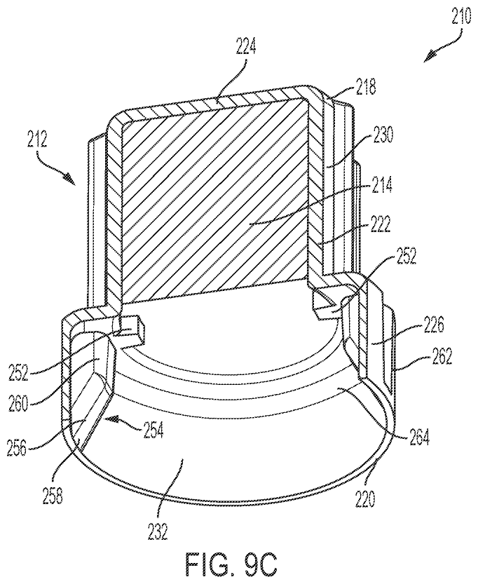

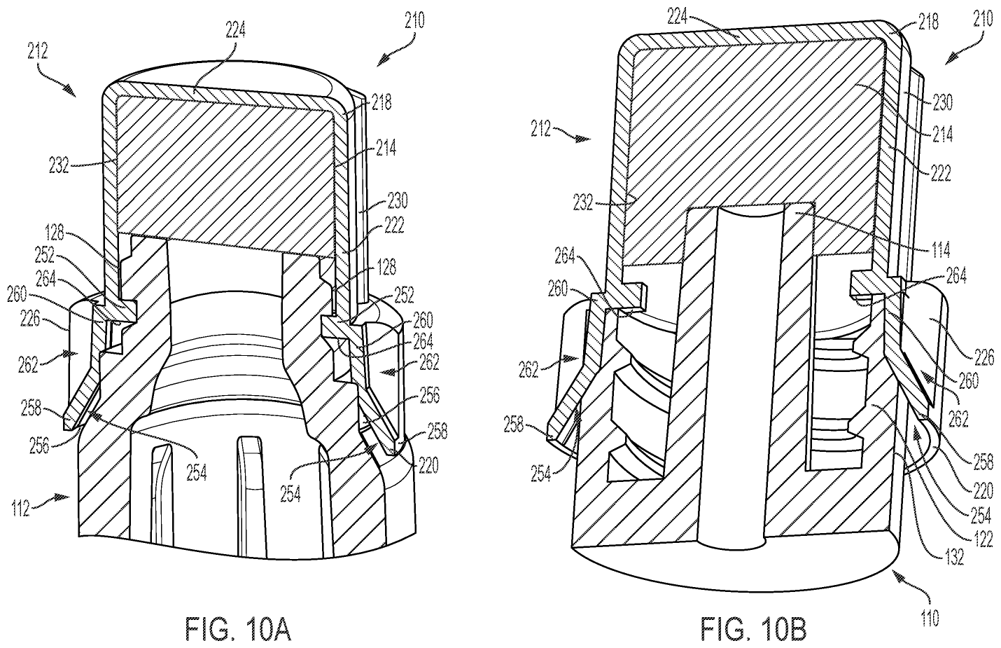

FIGS. 9A-9C show another example of a universal cap 210 configured to engage a female connector 112 and a male connector 110 including features of the present disclosure. FIG. 10A shows the cap 210 connected to a female connector 112. FIG. 10B shows the cap 210 connected to a male connector 110.

As in previous examples, the cap 210 comprises a housing 212, such as a cup-shaped housing, and an absorbent support 214 disposed in the housing 212. The housing 212 includes first or top end 218, which can be closed by a wall 224 or cover, an open second or bottom end 220, and a sidewall 222 extending between the top end 218 and the bottom end 220. As in previous examples, the absorbent support 214 is positioned in the housing 212 and is configured to contact portions of the male or female connector 110, 112 inserted into the housing 212. The cap 210 can also include a seal (not shown in FIGS. 9A-10B) mounted to a portion of the absorbent support 214 configured to cover an opening of the male or female connector 110, 112 when the connector 110, 112 is inserted into the housing 212.

The universal cap 210 differs from previous examples in the configuration of portions of the housing 212 that engage portions of the female connector 112 or the male connector 110. Specifically, the housing 212 of the cap 210 comprises one or more prongs 252 extending inwardly from an inner surface 232 of the housing 212. For example, the prongs 252 can be positioned on the inner surface 232 between a rigid portion 230 and a flexible portion 226 of the housing 212. The prongs 252 are configured to engage threads 128 of the female connector 112 for securing the female connector to the cap 210. The prongs 252 are provided as an alternative retention structure to the threads on the inner surface 32 of the housings 12 shown in other examples.

The housing 212 of the cap 210 also includes one or more ramp protrusions 254 extending inwardly from the inner surface 232 of the sidewall 222. For example, the ramp protrusions 254 can be positioned on the flexible portion 226 of the housing 212, as shown in FIGS. 9B and 9C. As shown in FIG. 9C, the ramp protrusions 254 can include a narrow side 258 proximate to the open bottom end 220 of the housing 212, a wide side 260 positioned in an interior of the housing 212 opposite the narrow side 258, and an angled or sloped surface 256 extending between the wide side 260 and the narrow side 258. In some examples, the ramp protrusions 254 can extend axially along the inner surface 232 of the sidewall 222 meaning that a longitudinal axis of the ramp protrusions 254 is substantially parallel (e.g., within about 10% of parallel) to a longitudinal axis of the housing 212.

The ramp protrusions 254 are configured to form a friction engagement with the male connector 110 for securing the male connector 110 to the cap 210. For example, the ramp protrusions 254 can be positioned to contact an outer surface 132 (shown in FIG. 10B) of the annular shield 122 creating the friction engagement between the outer surface 132 and the ramp protrusion 254. In some examples, the ramp protrusions 254 can be bendable or deformable, configured to bend or deform radially outward to accommodate annular shields 122 having different outer diameters. Further, the ramp protrusion 254 can be formed in the sidewall 222 of the housing 212 by an inwardly deformed portion of the sidewall 222, which provides an axial groove or slot 162 on an outer surface of the sidewall, as shown in FIGS. 9A and 9B. As previously described, the housing 212 can be a molded part made, for example, by an injection molding process. Therefore, the prongs 252 and/or ramp protrusions 254 can be integral with other portions of the housing 212 and formed along with the other portions of the housing 212 during the injection molding process.

In some examples, as shown in FIGS. 9B and 9C, the universal cap 210 can include two ramp protrusions 254 positioned on opposite sides of the housing 212. For example, the housing 212 can include a first ramp protrusion 254 positioned on one side of the housing 212 and a second ramp protrusion 254 positioned on the other side of the housing 212, about 180 degrees apart from the first ramp protrusion 254 about an inner circumference of the sidewall 222 of the housing 212. As shown in FIG. 9C, each prong 252 of the housing 212 can be axially aligned with one of the ramp protrusions 254 on the inner surface 232 of the housing 212.

The universal cap 210 is connected to either the male connector 110 or the female connector 112 in a similar manner to previous examples. Specifically, in order to connect the universal cap 210 to the female connector 112, the practitioner inserts the distal end of the female connector 112 through the open bottom end 220 of the housing 212. The female connector 112 is advanced into the housing 212 until threads 128 of the female connector 112 contact the prongs 252 extending inwardly from the inner surface 232 of the sidewall 222. The practitioner can then rotate the cap 210 relative to the female connector 112 causing the threads 128 to engage the prongs 252 by a rotational engagement, thereby fully securing the cap 210 to the female connector 112. A female connector 112 is shown fully inserted into and engaged with the universal cap 210 in FIG. 10A.

In order to connect the male connector 110 to the universal cap 210, the practitioner inserts the stem 114 and distal end of the annular shield 122 of the male connector 110 through the open bottom end 220 of the housing 212. The practitioner advances the male connector 110 into the housing 212 causing the outer surface 132 of the annular shield 122 of the male connector 110 to contact the ramp protrusions 254. If the annular shield 122 has a large outer diameter, the ramp protrusions 254 may bend or deform radially outward to permit insertion of the larger male connector 110 into the cap 210. The practitioner continues to move the male connector 110 into the cap 210 increasing the friction or interference engagement between the ramp protrusions 254 and the annular shield 122 until the male connector 110 is fully inserted into the cap 210 with a distal edge of the annular shield 122 in contact with an annular surface or lip 264 of the universal cap 210. The male connector 110 is shown fully inserted into the universal cap 210 in FIG. 10B.

While examples of the universal cap 10, 10b, 10c, 10d, 210 and methods of use of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A cap configured to engage at least a first connector and a second connector of different types, the cap comprising:
a housing comprising a first end, an open second end, and a sidewall extending between the first end and the second end;
an absorbent support positioned in the housing configured to contact portions of the first connector or the second connector inserted into the housing; and
a seal mounted to a portion of the absorbent support configured to cover an opening of the first connector or the second connector when one of the first connector or the second connector is inserted into the housing,
wherein the cap is configured to be secured to the first connector by an engagement between a portion of an inner surface of the sidewall of the housing and the first connector and to the second connector by an interference engagement between another portion of the inner surface of the sidewall of the housing and an outer surface of the second connector.

2. The cap of claim 1, wherein the first connector is a female luer connector comprising a threaded outer surface that engages the inner surface of the sidewall of the housing.

3. The cap of claim 2, wherein the inner surface of the sidewall comprises threads that engage the threaded outer surface of the female luer connector, and wherein the housing is sized to receive the female luer connector having an outer diameter of at least 12.5 mm.

4. The cap of claim 1, wherein the second connector comprises a male luer connector comprising a stem configured to be inserted into the absorbent support, such that the opening of the male luer connector is sealed by the seal mounted to the absorbent support.

5. The cap of claim 4, wherein the male luer connector further comprises an annular shield comprising a threaded inner surface extending about the stem, and wherein the housing is sized to receive the male luer connector having an outer diameter of from 8.0 mm to 14.0 mm.

6. The cap of claim 1, wherein the sidewall of the housing comprises a flexible portion proximate to the second end of the housing configured to press against and deform to accommodate portions of the first connector or the second connector when the first connector or the second connector is inserted into the housing.

7. The cap of claim 6, wherein the flexible portion of the sidewall is configured to extend radially outwardly to accommodate portions of the first connector or the second connector having an outer diameter larger than an inner diameter of the sidewall in an unbiased state.

8. The cap of claim 6, wherein the flexible portion deforms to form an interference engagement with the outer surface of the second connector.

9. The cap of claim 6, wherein the sidewall further comprises a rigid portion that does not deform when the first connector or the second connector is inserted into the housing.

10. The cap of claim 9, wherein the rigid portion comprises a threaded inner surface configured to engage a corresponding threaded outer surface of the first connector.

11. The cap of claim 1, wherein the housing is a single-molded part, comprising a rigid thermoplastic polymer comprising at least one of polyester, polycarbonate, polypropylene, polyethylene, polyethylene terephthalate, or acrylonitrile butadiene styrene.

12. The cap of claim 1, wherein the absorbent support comprises a cylindrical member sized to be received within a substantially cylindrical cavity defined by a portion of the inner surface of the sidewall of the housing.

13. The cap of claim 1, wherein the absorbent support comprises a porous foam comprising a thermoplastic elastomeric material.

14. The cap of claim 1, wherein insertion of the first or second connector into the cap causes the connector to contact the absorbent support, and wherein contact between the connector and the absorbent support mechanically removes objects from an outer surface of the connector.

15. The cap of claim 1, further comprising a cleaning or disinfecting solution absorbed by the absorbent support, wherein the cleaning or disinfecting solution comprises from about 0.5% to about 3.5% chlorhexidine gluconate and about 70% IPA.

16. The cap of claim 1, wherein the seal comprises a closed cell foam.

17. The cap of claim 1, further comprising a removable protective cover attached to the open second end of the housing for protecting an interior of the housing and the absorbent support prior to use.

18. The cap of claim 1, wherein the absorbent support is shaped to clean and/or disinfect a distal portion and outer surface of a female connector and a tip and outer surface of a stem and inner surface of an annular shield of a male connector.

19. The cap of claim 1, further comprising at least one ramp protrusion extending inwardly from the inner surface of the housing positioned to contribute to the engagement between the second connector and the cap.

20. A cap configured to engage at least a first connector and a second connector of different types, the cap comprising:
a housing comprising a first end, an open second end, a sidewall extending between the first end and the second end, at least one prong extending inwardly from an inner surface of the sidewall, and at least one ramp protrusion extending inwardly from the inner surface of the sidewall;
an absorbent support positioned in the housing configured to contact portions of the first connector or the second connector inserted into the housing; and
a seal mounted to a portion of the absorbent support configured to cover an opening of the first connector or the second connector when one of the first connector or the second connector is inserted into the housing,
wherein the at least one prong is configured to engage threads of the first connector for securing the first connector to the cap, and the at least one ramp protrusion is configured to form a friction engagement with the second connector for securing the second connector to the cap.
